(19) United States
(12) Patent Application Publication
(10) Pub. No.: US 2025/0195730 A1
(43) Pub. Date: Jun. 19, 2025

(54) GENERATING MEDICAL FLUID FOR RENAL REPLACEMENT THERAPY

(71) Applicant: GAMBRO LUNDIA AB, Lund (SE)

(72) Inventors: Olof JANSSON, Vellinge (SE); Per-Olof BORGQVIST, Lund (SE); Michael PETTERSSON, Malmö (SE); Jonas FORS, Malmö (SE)

(21) Appl. No.: 18/844,270

(22) PCT Filed: Jan. 24, 2023
(86) PCT No.: PCT/EP2023/051676

§ 371 Date: Sep. 5, 2024

(30) Foreign Application Priority Data
Mar. 16, 2022 (SE) 2250327-0

(51) Int. Cl. A61M 1/14; A61M 1/16; A61M 1/28; A61M 1/34

(52) U.S. Cl. CPC A61M 1/154; A61M 1/1605; A61M 1/282; A61M 1/3406; A61M 2205/273; A61M 2205/3393

(57) ABSTRACT

A system for generating a medical fluid for renal replacement therapy is operated by a control device according to a method. In the method, a first pump is operated to convey a first fluid from a first container, arranged on a first scale, into a supply path, and a second pump is operated to convey a second fluid from a second container, arranged on a second scale, into the supply path, to generate a mixture therein. The speed of at least one of the pumps is adjusted until a sensor measures a target value of a composition-related parameter. A relation between weight changes of the first and second scales is determined while the sensor measures the target value. The medical fluid is then generated in the supply path, by the pumps being operated, based on signals from the scales, to achieve said relation.

### TECHNICAL FIELD

The present disclosure relates to the field of renal replacement therapy and in particular to generation of a medical fluid for use in such therapy.

### BACKGROUND ART

Renal replacement therapy (RRT) is a therapy that replaces the normal blood-filtering function of the kidneys. It is used when the kidneys are not working well, which is known as kidney failure and includes acute kidney injury (AKI) and chronic kidney disease (CKD). RRT involves removal of water from the blood of the patient suffering from kidney failure, as well as exchange of solutes with the blood. One example of RRT is extracorporeal blood therapy, in which blood is circulated outside of the patient and interfaced with one or more medical fluids. Modalities of extracorporeal blood therapy include hemodialysis (HD), hemofiltration (HF) and hemodiafiltration (HDF). Another example of RRT is peritoneal dialysis (PD), in which a medical fluid is infused into the peritoneal cavity of the patient to interface with the blood of the patient through the peritoneal membrane.

Medical fluids used in HD and PD are commonly known as dialysis fluids. In HF, the medical fluid is known as replacement fluid, since it is infused into the blood of the patient to replace fluid removed during therapy. In HDF, both dialysis fluid and replacement fluid are used.

Extracorporeal blood therapy by HD, HF or HDF is performed differently for treatment of patients with AKI compared to patients with CKD, by use of a different type of dialysis machine. Generally, compared to CKD patients, AKI patients are treated continuously over a longer period of time and at lower fluid flow rates. Such continuous treatment is commonly known as CRRT (Continuous Renal Replacement Therapy). To ensure precise and consistent monitoring and control of fluid removal, known as ultrafiltration, AKI machines are typically provided with scales that are used for measuring the weight of fresh treatment fluid and the weight of spent treatment fluid during therapy. CKD machines instead use flow meters or volumetric pumping to control ultrafiltration.

PD machines, also known as cyclers, may include at least one scale to measure the weight of fresh treatment fluid infused into the peritoneal cavity and the weight of spent treatment fluid withdrawn from the peritoneal cavity.

Over time, RRT consumes large quantities of medical fluid. In some modalities of RRT, pre-made medical fluid is delivered in prefilled bags to the point of care. For example, conventional PD is performed by use of prefilled bags. AKI machines are configured to use prefilled bags of medical fluid, by staff arranging a prefilled bag on one of the scales before treatment, and replacing the prefilled bag as required. On the other hand, CKD machines have integrated capability to generate medical fluid on-demand by mixing one or more concentrates with water, so-called on-line fluid generation. Recently, PD machines with integrated capability of on-line fluid generation have been proposed.

The use of large quantities of medical fluid has significant environmental impact through transportation and the large amount of plastic material that is consumed to produce the prefilled bags. In an intensive care unit, the administration and handling of prefilled bags at the point of care is taxing on the staff, takes time and diverts the attention of the staff from other tasks. There is thus a general need to enable on-line generation of medical fluid also in AKI machines, and preferably without the need to replace existing AKI machines.

### SUMMARY

It is an objective to at least partly overcome one or more limitations of the prior art.

A further objective is to enable on-line generation of a medical fluid by a dialysis machine that comprises a plurality of scales.

One or more of these objectives, as well as further objectives that may appear from the description below, are at least partly achieved by a method of generating a medical fluid for renal replacement therapy, a control device, a computer-readable medium, a system for generating a medical fluid, and a disposable arrangement, embodiments thereof being defined by the dependent claims.

A first aspect is a computer-implemented method of generating a medical fluid for renal replacement therapy. The method comprises: operating a first pump to convey a first fluid from a first container, which is arranged on a first scale, into a supply path that extends to an outlet for the medical fluid; operating a second pump to convey a second fluid from a second container, which is arranged on a second scale, into the supply path, to generate a mixture of the first and second fluids in the supply path; measuring, by a sensor, a composition-related parameter of the mixture; and adjusting a pumping speed of at least one of the first and second pumps until the sensor measures a target value of the composition-related parameter. The method further comprises: determining, based on first and second output signals from the first and second scales, a relation between a first weight change of the first scale and a second weight change of the second scale while the sensor measures the target value; and generating the medical fluid in the supply path. The step of generating comprises: operating, based on the first and second output signals, the first and second pumps to achieve the relation between the first and second weight changes.

A second aspect is a control device. The control device comprises circuitry, which is configured to perform the method of the first aspect, and a signal interface, which is configured to output control signals for the first and second pumps. The signal interface is further configured to receive the first and second output signals from the first and second scales and a sensor signal representative of the composition-related parameter from the sensor.

A third aspect is a computer-readable medium comprising computer instructions which, when executed by a processor, cause the processor to perform the method of the first aspect.

A fourth aspect is a system for generating a medical fluid for renal replacement therapy. The system comprises a first scale, a first container arranged on the first scale, a second scale, and a second container arranged on the second scale. The system further comprises a supply path, which is configured to receive a first fluid from the first container and a second fluid from the second container and extends to an outlet for the medical fluid, a first pump arranged to convey the first fluid from the first container into the supply path, a second pump arranged to convey the second fluid from the second container into the supply path to generate a mixture of the first and second fluids in the supply path, a sensor configured to measure a composition-related parameter; and the control device of the second aspect.

A fifth aspect is a disposable arrangement for use in the system of the fourth aspect. The disposable arrangement comprises: the first container, the supply path, and a connecting line, which is in fluid communication with the supply path and extends to a first terminating fluid connector, which is configured for connection to the second container. The disposable arrangement further comprises the bypass path, which extends from the supply path to a second terminating fluid connector, which is configured for connection to an inlet connector in fluid communication with the sensor.

Still other objectives and aspects, as well as embodiments, features and technical advantages, may appear from the following detailed description, from the attached claims as well as from the drawings.

### BRIEF DESCRIPTION OF THE DRAWINGS

FIG. 1 is a schematic diagram of an example system for dialysis therapy.

FIGS. 2A-2B are schematic diagrams of a disposable arrangement and a machine part, respectively, for use in the system of FIG. 1.

FIGS. 3A-3B shows the system of FIG. 1 during on-going therapy and in an intermediate renewal phase, respectively.

FIG. 4 is a flow chart of an example method of performing fluid control in the system of FIG. 1.

FIGS. 5A-5B show the system of FIG. 1 during a first and second preparation phase, respectively, in the method of FIG. 4.

FIGS. 6-7 depict variants of the system in FIG. 1.

FIG. 8 is a flow chart of an example method of operating the system of FIG. 1.

FIG. 9 is a schematic diagram of an example water supply device comprising a re-usable sensor.

FIGS. 10A-10B are side views of a re-usable sensor before and after manipulation of a disposable line set to encase a fluid within the sensor.

FIG. 11 is a flow chart of an example procedure of performing fluid control during interruption of dialysis therapy.

FIG. 12 is a schematic diagram of an example system for generating medical fluid.

FIGS. 13A-13B are flowcharts of example procedures for use of an auxiliary sensor.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more,” even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

It will furthermore be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure. As used herein, the terms “multiple”, “plural” and “plurality” are intended to imply provision of two or more elements. The term “and/or” includes any and all combinations of one or more of the associated listed elements.

Well-known functions or structures may not be described in detail for brevity and/or clarity. Unless otherwise defined, all terms (including technical and scientific terms) used herein have the same meaning as commonly understood by one of ordinary skill in the art to which this disclosure belongs.

The present disclosure relates to a technique for on-line generation of medical fluid for use in renal replacement therapy (RRT). As used herein, RRT refers to any therapy that replaces or supplements the normal blood-filtering function of the kidneys in a patient. RRT may involve removal of water from the blood of the patient, as well as exchange of solutes with the blood. The technique for on-line fluid generation will be exemplified in the following with reference to hemodialysis (HD), although it is applicable to any modality of either extracorporeal blood therapy or peritoneal dialysis (PD).

As used herein, on-line fluid generation refers to the generation of medical fluid on the fly, by mixing various constituents in adequate proportions. On-line fluid generation may comprise on-demand fluid generation, in which the production rate of the medical fluid is adjusted to match the consumption rate of the medical fluid, for example by on-going RRT. It is also conceivable to implement the on-line fluid generation to be independent of the consumption rate of the medical fluid, for example by providing the generated medical fluid to a storage vessel or reservoir.

The medical fluid may be any fluid that is consumed as part of RRT and is also referred to as “treatment fluid”. In the context of extracorporeal blood therapy, the medical fluid may be a dialysis fluid, which is interfaced with blood in a filtration unit, commonly known as a “dialyzer”. Alternatively or additionally, the medical fluid may be a so-called replacement fluid or substitution fluid, which is infused into the blood upstream or downstream of the dialyzer, for example as part of hemofiltration (HF) or hemodiafiltration (HDF), as is well known in the art. In the context of PD, the medical fluid may be a dialysis fluid, which is infused into the peritoneal cavity of the patient and which interfaces with the blood of the patient through the peritoneal membrane that lines the peritoneal cavity.

Medical fluids for use in RRT have well-defined compositions, which are tailored to the specific therapy and to the patient. In on-line generation, the composition of the medical fluid may be given by medical guidelines and/or be set by a caretaker for a specific patient or group of patients. Medical guidelines may also define allowable deviations from nominal concentrations of various solutes in the medical fluid.

As used herein, a “disposable device” (or part/arrangement/sensor, etc.) is a device that is intended to be used only for a limited time, after which the device is replaced for a new disposable device. Depending on disposable device, the limited time may correspond to a single treatment session or a predefined number of treatment sessions, or be given by a predefined maximum time period. In another alternative, a disposable device may be replaced whenever a new patient is to be treated.

As used herein, a “re-usable device” (for example, a sensor) is a device that has a longer operative life than a corresponding device that is disposable. In some embodiments, the re-usable device is not intended to be replaced at all. Thus, its operative life is endless, at least in theory. Colloquially, such a device is often referred to as being “permanent” or “permanently installed”, although the device will be replaced if it is deemed to be malfunctioning.

As used herein, a “treatment session” or “session” refers to a time period during which a patient is subjected to RRT by use of a dialysis machine. The time period starts when the patient is connected to the dialysis machine and ends when the patient is disconnected from the dialysis machine.

In the technique proposed herein, the medical fluid is generated as a mixture of two or more constituent fluids. The constituent fluids are provided from a respective container arranged on a respective scale. The technique is based on the insight that it may be advantageous to control the supply of constituent fluids based on measurement signals from the scales. Such control is possible if a mixing ratio of the constituent fluids is known. While the mixing ratio may be theoretically determined based on the nominal compositions of the constituent fluids, the use of a theoretical mixing ratio may introduce unacceptable deviations in the composition of the medical fluid, for example if the actual composition of a constituent fluid deviates from its nominal composition or if the wrong constituent fluid is installed on a scale in the dialysis machine. Thus, the use of a theoretical mixing ratio may require strict tolerances for the composition of the respective constituent fluid and may also require implementation of extraordinary measures to prevent human error when installing the containers on the scales.

An alternative approach would be to control the supply of the respective constituent fluid based on a feedback signal generated by a sensor which is arranged to measure a composition-related parameter (CRP) of the medical fluid, for example conductivity. Such a CRP sensor is thus arranged in the main flow path for the medical fluid, upstream of the dialyzer, to provide continuous feedback about the current value of the CRP while the medical fluid is being generated. This approach is often used in CKD machines with integrated capability of on-line fluid generation, as discussed in the Background section. One drawback of this approach is that the CRP sensor intermittently needs to be thoroughly disinfected, for example after each treatment session. Since the CRP sensor is located in the main flow path for the medical fluid, it is imperative to prevent microorganisms from growing in the CRP sensor, since such microorganisms would be carried over into the medical fluid and potentially harm the patient. To obviate the need for disinfection, the CRP sensor may instead be installed as a disposable unit, which is discarded after use, for example after each treatment session. However, this would increase the cost of treatment considerably. For example, conductivity sensors of sufficient accuracy are quite costly.

All of these drawbacks would be overcome if the mixing could instead be controlled based on measurement signals from the scales, in view of a mixing ratio. The technique proposed herein derives mixing control data (MCD) by use of a CRP sensor in an arrangement described below. The MCD accounts for the actual compositions of the constituent fluids and designates a desired relation between the measurement signals from the scales. The MCD thereby replaces the theoretical mixing ratio and obviates the associated drawbacks. At the same time, by use of a clever tuning procedure for deriving the MCD, the need for intermittent replacement of the CRP sensor is also obviated. As will be described in detail further below, the tuning procedure involves directing a mixture of two constituent fluids, from a respective container on a respective scale, through a CRP sensor while adjusting the supply rate of at least one of the constituent fluids. When the CRP sensor measures a target value, which corresponds to a desired mixing ratio between the constituent fluids, the current relation between the weight changes of the scales is determined. The tuning procedure may be repeated for further constituent fluids, if included in the medical fluid, resulting in a set of relations that results in the desired mixing ratio of the constituent fluids in the medical fluid. When the medical fluid is to be generated and provided for use in RRT, the supply of the constituent fluids is controlled to achieve the relation(s) between the weight changes of the scales, thereby inherently producing the medical fluid with the desired composition. Since the supply of the constituent fluids is controlled based on measurement signals from the scales, there is no need for continuous feedback from the CRP sensor while the medical fluid is being generated. This means that the CRP sensor may be arranged to only be exposed to the constituent fluids during the tuning procedure. In other words, the medical fluid that is provided for use in the RRT need not pass through the CRP sensor and is thus not exposed to any microorganisms that may be present in the CRP sensor. Thereby, the need for intermittent disinfection of the CRP sensor is mitigated, as well as the need of intermittently replacing the CRP sensor. Further, by limiting its exposure to the medical fluid, fouling of the CRP sensor is reduced, for example in terms of scaling. Thereby, the operative life of the CRP sensor is extended, and it may even be possible to use a permanently installed CRP sensor. Since CRP sensors generally are expensive, significant cost savings are possible.

As noted in the Background section, commercially available dialysis machines for use in treatment of acute kidney injury (AKI) typically comprise scales for controlling dialysis flow parameters. As will be understood from the following, such existing dialysis machines may be simply re-configured to use the technique proposed herein. Thus, dialysis machines that are designed to use bags of pre-made medical fluid may be converted to produce the medical fluid on-line. Of course, it is also conceivable that novel dialysis machines are developed to utilize the technique proposed herein, for use in any modality of extracorporeal blood therapy or PD.

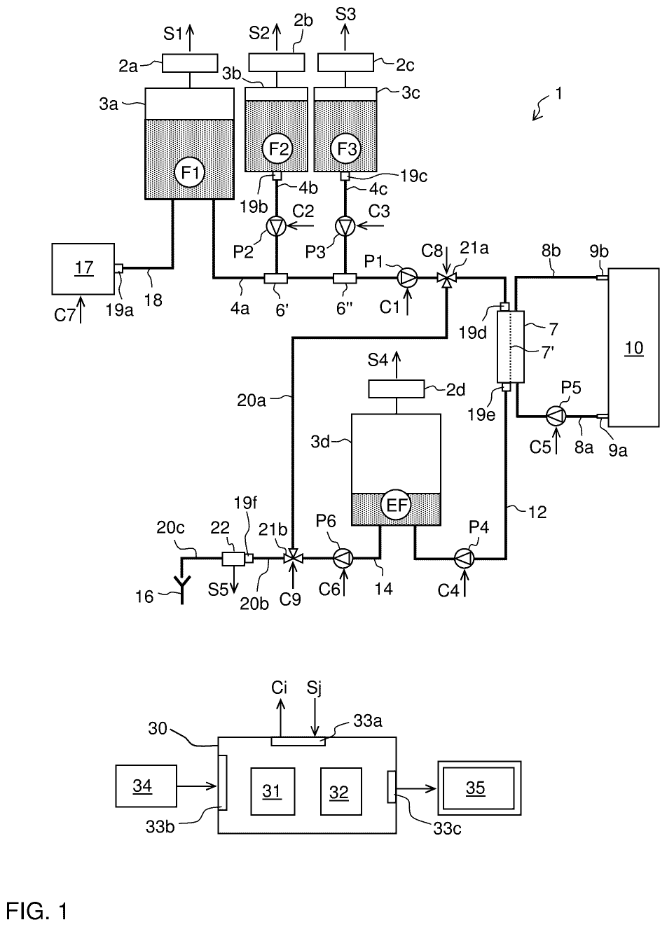

Various embodiments will now be described with reference to FIG. 1, which illustrates an example system 1 for dialysis therapy, specifically a system for extracorporeal blood therapy by hemodialysis. The system 1 is configured to generate a dialysis fluid on-demand for use in hemodialysis. The system 1 comprises scales 2a-2d which are configured to provide a respective measurement signal S1-S4 representing mass. A container 3a-3d is arranged on the respective scale 2a-2d. Instead being hung from the scales 2a-2d, as shown, the containers 3a-3d may be placed to rest on the scales 2a-2d. The containers 3a-3d may be rigid or flexible and may be of any material, for example plastics. In the illustrated example, the dialysis fluid is generated by mixing three fluids F1, F2, F3 (“constituent fluids”). The first fluid F1 in the first container 3a is water, and the second and third fluids F2, F3 in the second and third containers 3b, 3c are different liquid concentrates. For example, the concentrates may be so-called A and B concentrates, as known in the art. In the following, it is assumed that F2 is an A concentrate and F3 is a B concentrate, although the opposite is equally possible. The system 1 further comprises a pumping arrangement comprising pumping devices (“pumps”) P1-P6. The respective pump P1-P6 may be of any type. If the fluid paths in the system are defined by a disposable arrangement (below), the pumps P1-P6 are typically peristaltic pumps, which engage the outside of dedicated tubing. The pumps P1-P6 are operated in response to control signals C1-C6.

A first fluid line 4a extends from the first container 3a to a dialyzer 7 and forms a main supply path for the dialysis fluid. A second fluid line 4b extends from the second container 3b to a first junction 6′ on the first fluid line 4a, and a third fluid line 4c extends from the third container 3c to a second junction 6″ downstream of the first junction 6′ on the first fluid line 4a. The system 1 is configured to allow a flow of F2 to meet and form a mixture with a flow of F1 in the first fluid line 4a, within and downstream of junction 6′. Similarly, the system 1 is configured to allow a flow of F3 to meet and form a mixture with the combined flow of F1 and F2 in the first fluid line 4a, within and downstream of junction 6″. The mixing of F1 and F2 may or may not be completed at junction 6″, but the mixing of F1, F2 and F3 is completed at the outlet from first fluid line 4a, i.e. on entry into the dialyzer 7. In the illustrated example, the pump P1 is arranged in the first fluid line 4a to define the flow rate of dialysis fluid (“main flow rate”), and the pumps P2, P3 are arranged in the second and third fluid lines to define the flow of F2, F3, respectively. Thus, the pumps P2, P3 are operable to control the amounts of F2, F3 admixed into F1 within the first fluid line 4a.

Although not shown in FIG. 1, the system 1 may include one or more devices configured to promote the mixing, for example inside the respective junction 6′, 6″. In some embodiments, the respective junction 6′, 6″ is a 3-way connector. A non-limiting example of a 3-way connector with a mixing-enhancement device is disclosed in WO2009/030973, which is incorporated herein by reference. In a variant, the mixing-enhancement device may be separate from and located downstream of junction 6′, 6″. Such a separate device may be configured to increase the Reynolds number of the mixture and/or at least one of the incoming fluid flows. Alternatively, the separate device may be configured as a conventional static mixer, or a recirculation circuit in which the mixture is circulated to promote mixing before being conveyed to the dialyzer 7.

The dialyzer 7 is a conventional blood filter, in which a semipermeable membrane 7′ is arranged to define a first chamber for dialysis fluid and a second chamber for blood. One end of the first chamber is connected to receive the dialysis fluid from the first fluid line 4a. The dialysis fluid flows through the first chamber and leaves the dialyzer 7 at the opposite end. The dialysis fluid that leaves the dialyzer 7 is “spent”. To distinguish fresh dialysis fluid from spent dialysis fluid, the latter is denoted “effluent” herein and designated by EF. The second chamber of the dialyzer 7 is arranged to receive blood from a withdrawal line 8a, which is connected by a connector 9a to a subject 10. Typically, the subject 10 is a dialysis patient, and the connector 9a is an access device (catheter, needle, etc.) in fluid communication with the circulatory system of the patient. However, it also conceivable that the subject 10 is a reservoir of blood to the treated. The second chamber is also in fluid communication with a return line 8b, which extends to a connector 9b, which is connected to the subject 10. The pump P5 is arranged along the withdrawal line 8a to pump blood from the subject or patient 10 through the withdrawal line 8a, the second chamber, and the return line 8b back to the subject 10. When passing the second chamber, the blood is interfaced with the dialysis fluid through the membrane 7′ and thereby treated by hemodialysis. The principle of hemodialysis is well-known to the skilled person and will not be further explained herein. It should be appreciated that the example in FIG. 1 is simplified and that further conventional components may be included, such as clamps, pressure sensors, air detector, etc.

In the example of FIG. 1, the first chamber of the dialyzer 7 is fluidly connected to an effluent line 12, which extends to the fourth container 3d. The pump P4 is arranged along the effluent line 12 to pump EF from the dialyzer 7 into the container 3d. The container 3d is further fluidly connected to a drain line 14. The drain line 14 is part of a drain path that extends to a drain 16. The pump P6 is arranged along the drain line 14 to pump EF from the container 3d to the drain 16. The pump P6 is operable to completely or at least partly drain the container 3d, during a drain phase. As will be described below, the drain phase may be selectively initiated when the container 3d is deemed to be (sufficiently) full.

In the illustrated example, the drain path comprises the drain line 14 and an adjoining line segment 20b, which is connected to a sensor 22, and a further adjoining line segment 20c, which extends from the sensor 22 to the drain 16. The sensor 22 is configured to measure a composition-related parameter (CRP) of the passing fluid. The CRP may represent conductivity, or equivalently resistivity. In a variant, the CRP represents the concentration of a substance in the fluid, specifically a substance that is present in fresh dialysis fluid, for example bicarbonate or an electrolyte such as sodium, potassium, calcium, magnesium, chloride, etc. If the dialysis fluid is generated for use in PD, the substance may alternatively be an osmotic agent such as glucose. In a further alternative, the CRP may represent the concentration of hydrogen ions, for example in the form of a pH value. As shown, the sensor 22 provides a measurement signal S5, which is indicative of the CRP.

As noted, the first fluid F1 is water, which is the major constituent in the dialysis fluid. To sustain continued generation of the dialysis fluid the container 3a may need to be replenished. To this end, the system 1 comprises a water supply device 17, which is configured to generate water of sufficient quality for use in dialysis fluid. The supply device 17 is connected to the container 3a by a water supply line 18. The supply device 17 is operable to supply water to the container 3a, subject to a control signal C7, to completely or at least partly refill the container 3d during a refill phase. As will be described below, the refill phase may be selectively initiated when the container 3a is deemed to be (sufficiently) empty.

In FIG. 1, a bypass line 20a is arranged to extend from the first fluid line 4a to the drain path. The system 1 further includes a valve arrangement, which is operable in different states. In a first state, fluid flow is directed along the first fluid line 4a to the dialyzer 7. In a second state, fluid flow is prevented from flowing to the dialyzer 7 and is instead diverted into the bypass line 20a and directed from the bypass line 20a into the drain path towards the drain 16. Thus, in the second state, a “bypass path” is defined that comprises the bypass line 20a, and the line segments 20b, 20c. In a third state, EF is selectively allowed to flow from the container 3d along the drain path to the drain 16, i.e. through the drain line 14, and the line segments 20b, 20c. The valve arrangement is not operable in the first and second states at the same time (mutually exclusive), nor in the second and third states. Depending on implementation, the valve arrangement may or may not be operable in the first and third states at the same time. In the illustrated example, the valve arrangement comprises first and second valve devices 21a, 21b, which are arranged at the upstream end and the downstream end, respectively, of the bypass line 20a. The respective valve device 21a, 21b may be a three-way valve, as shown, or a combination of on/off valves. The state of the valve arrangement is set by control signals C8, C9 for the first and second valve devices 21a, 21b. If the fluid paths in the system are defined by a disposable arrangement (below), the valve arrangement may comprise actuators that are configured to engage the outside of dedicated tubing to selectively close off fluid passages. Such a valve arrangement may comprise pinch valves or clamps of any type.

As shown in FIG. 1, a control device 30 is configured to control the operation of any system described herein. If the system 1 is operated by a dialysis machine, the control device 30 may be a controller of the dialysis machine or a separate controller. The control device 30 is configured to receive measurement signals, represented as Sj, and output control signals, represented as Ci. As understood, Ci may include C1-C9, and Sj may include S1-S5. The control device 30 may be configured to generate the control signals Ci in accordance with a control program comprising computer instructions. The control program is also configured to operate based on the measurement signals Sj. The control device 30 comprises circuitry that includes one or more processors 31 and computer memory 32. The control program is stored in the memory 32 and executed by the processor(s) 31. The control program may be supplied to the control device 30 on a computer-readable medium, which may be a tangible (non-transitory) product (e.g., magnetic medium, optical disk, read-only memory, flash memory, etc.) or a propagating signal. In the illustrated example, the control device 30 comprises a signal interface 33a for providing control signals Ci and receiving measurement signals Sj. The control device 30 also comprises an input interface 33b for connection to one or more input devices 34 that enable an operator to input control data, as well as an output interface 33c for connection to one or more UI devices 35 for providing feedback data to the operator. For example, the input device(s) 34 may comprise a keyboard, keypad, computer mouse, control button, touch screen, printer, microphone, etc., and the UI device(s) 35 may comprise a display device, a touch screen, an indicator lamp, an alarm device, a speaker, etc. The operator may be a clinically experienced person, such as a physician or a nurse, or the patient.

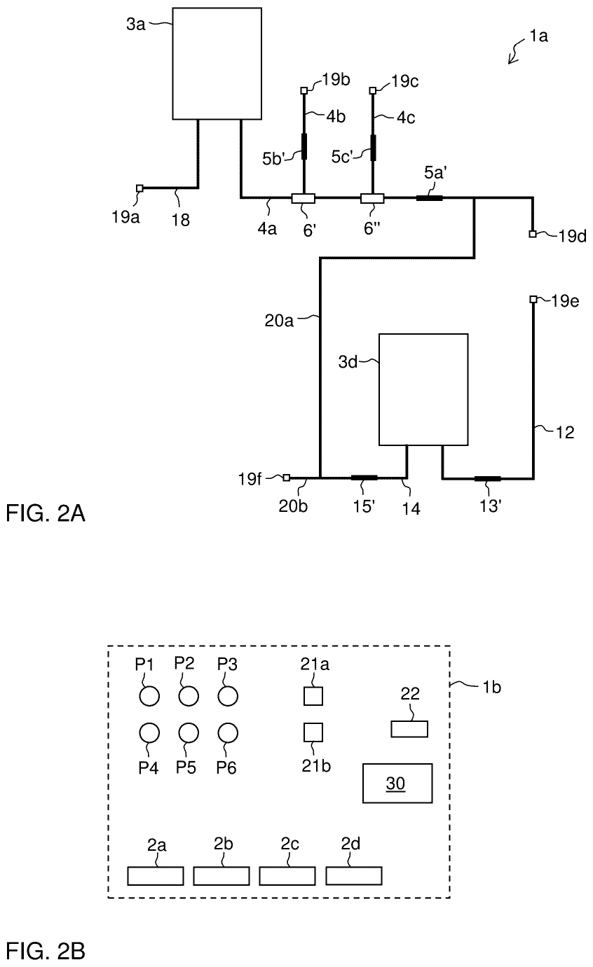

The system 1 in FIG. 1 also comprises a plurality of connectors 19a-19f attached to various fluid lines. The provision of the connectors 19a-19f enables the system 1 to comprise a disposable part or “disposable arrangement”, which is connectable to corresponding connectors in the system and is discarded after use. The disposable part is engaged with a machine part to form at least part of the system. In a dialysis system, the machine part is commonly referred to as a dialysis machine or a “monitor”. The disposable part may differ depending on the modality of treatment to be performed. An example of a disposable part 1a for HD therapy is shown in FIG. 2A. The disposable part 1a may be a unitary component, as shown, or an aggregation of connectable sub-parts. The disposable part 1a is provided as a dedicated accessory for installation on the machine part. Although not shown in FIG. 2A, the disposable part 1a may also include one or more of the containers 3b, 3c, which would be full of liquid concentrate when delivered. As is well-known in the art, the dialyzer 7 and the blood lines 8a, 8b are also disposables and may or may not be included in the disposable part 1a.

In FIG. 2A, the disposable part 1a defines fluid paths on the “non-blood side” of the system. Specifically, the disposable part 1a comprises the water supply line 18, the container 3a, the fluid lines 4a, 4b, 4c, the junctions 6′, 6″, the bypass line 20a, the effluent line 12, the container 3d, the drain line 14 and the line segment 20b. Connector 19a is arranged on the end of line 18 for connection to the supply device 17, connector 19b is arranged on the end of line 4b for connection to container 3b, connector 19c is arranged on the end of line 4c for connection to container 3c, connector 19d is arranged on the end of line 4a for connection to an inlet to the second chamber of the dialyzer 7, connector 19e is arranged on the end of effluent line 12 for connection to an outlet of the second chamber of the dialyzer 7, and connector 19f is arranged on the end of line segment 20b for connection to CRP sensor 22. The provision of connectors 19b, 19c makes it possible for an operator to remove the containers 3b, 3c when empty and re-attach new containers 3b, 3c full of concentrates F2, F3. The disposable part 1a is discarded after use, for example after completion of a treatment session, after a predetermined maximum time of use, or whenever a new patient is to be treated.

In the disposable part 1a, the fluid lines may be defined by plastic tubing. As noted above, the pumps P1-P6 may be peristaltic pumps which engage the outside of the tubing to generate a moving compression of the tubing to force fluid to move along the tubing. Conventionally, to enable the use of a peristaltic pump, the tubing is provided with a dedicated engagement portion, also known as a pump segment, which is configured to be engaged by compression element(s) of the peristaltic pump. In FIG. 2A, pump segments 5a′, 5b′, 5c′ are configured to be engaged by pumps P1, P2, P3 (FIG. 1), and pumps segments 13′, 15′ are configured to be engaged by pumps P4, P6 (FIG. 1).

The disposable part 1a may be engaged with the machine part 1b shown in FIG. 2B. In the illustrated example, the machine part 1b comprises the pumps P1-P6, which are accessible for engagement with the pump segments of the disposable part 1a. The machine part 1b also comprises the valve devices 21a, 21b, which are accessible for engagement with fluid lines in the disposable part 1a. The scales 2a-2d are also included in the machine part 1b and configured to carry the respective container 3a-3d. The machine part 1b also includes the control device 30 and thereby forms an operative apparatus for performing RRT. In the example of FIG. 2B, the machine part 1b also includes the CRP sensor 22, although alternative placements are possible (below).

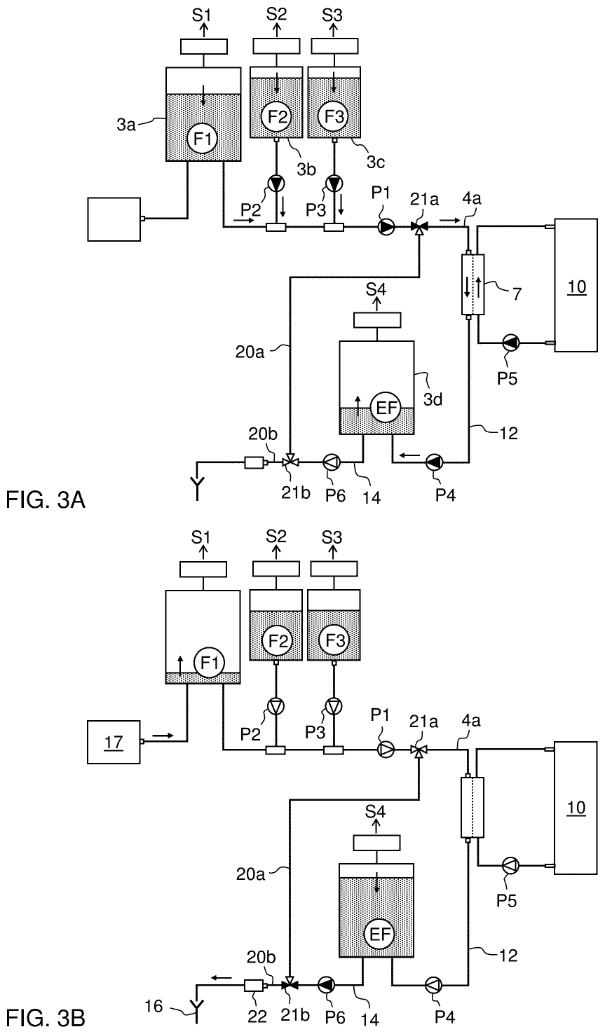

FIG. 3A shows the system 1 when operated, by the control device 30 (FIG. 1), to perform HD therapy. In FIG. 3A, and all other drawings, open/filled pump symbols represent a pump that is stopped/operating, and open/filled valve symbols represent a valve path that is closed/open. In FIG. 3A, pumps P1-P3 are jointly operated to pump water (F1) and concentrates (F2, F3), in well-defined proportions, into the supply line 4a while the valve arrangement is set in its first state to direct the fluids F1-F3 into the first chamber of the dialyzer 7. In this process, water and concentrates are mixed into a dialysis fluid within line 4a. Concurrently, pump P5 is operated to pump blood through the second chamber of the dialyzer 7. Pump P4 is also operated to pump effluent from the dialyzer 7 into the container 3d. By proper operation of pumps P1-P4 fluid may be extracted from blood in the dialyzer 7 through the membrane 7′. This extraction of fluid is known as ultrafiltration, and the extracted fluid is denoted ultrafiltrate. The rate of ultrafiltration at any given time is given by the difference in flow rate between pump P4 and pump P1, i.e. the outflow of effluent from the dialyzer 7 and the inflow of dialysis fluid into the dialyzer 7. Both the rate of ultrafiltration (UFR) and the accumulated amount of ultrafiltrate (UF) may be monitored by use of signals S1-S4. UFR is equal to the difference between the weight increase per unit time of container 3d and the sum of weight decrease per unit time of containers 3a-3c. UF for a time period is given by the difference between the weight increase of container 3d and the sum of weight decreases of containers 3a-3c during the time period. In some embodiments, the control device 30 is configured to control pump P1 and/or pump P4 based on the output signals S1, S4 to achieve a set value of UFR and/or UF.

It is realized that the system 1 is operated to generate the dialysis fluid on-line and on-demand. Further, the dialysis fluid is generated within the disposable part 1a. This means that an existing dialysis machine with scales may be configured for on-line generation of dialysis fluid by attachment of a properly configured disposable part 1a and by re-configuring the control device (30 in FIG. 1), for example by updating its control program.

During dialysis therapy, the fluid levels in containers 3a, 3b and 3c decrease and the fluid level in container 3d increases, as indicated by arrows. Eventually, container 3a will be depleted of water, resulting in a need to replenish the container 3a by initiating the above-mentioned refill phase. Likewise, container 3d will eventually be full of effluent (EF), resulting in a need to drain the container 3d by initiating the above-mentioned drain phase. The control device 30 may monitor the need for replenishment and draining based on the weight of the respective container 3a, 3d, given by signals S1, S4. FIG. 3B shows the system 1 during refill and drain phases, which in this example are performed concurrently. In the refill phase, the supply device 17 is operated to supply water, resulting in an increasing fluid level in the container 3a. In the drain phase, the pump P6 is operated to pump EF from the container 3d into the drain path and the valve arrangement is set in its third state to direct EF to drain 16. In the illustrated example, dialysis therapy is interrupted during the drain and refill phases, by pumps P1-P5 being stopped. However, pump P5 may be operating to avoid clogging in the blood lines. In an alternative, which is a superposition of FIGS. 3A and 3B, the dialysis therapy is continued during the drain phase and/or the refill phase. It is realized that the UFR and UF cannot be reliably monitored or controlled by use of the signals S1-S4 during such drain and refill phases.

The liquid concentrates F2, F3 will be consumed at much lower rate than the water F1. When the control device 30 detects, based on signals S2, S3, that one of the containers 3b, 3c is getting empty, an alert may be generated on the UI device 35 (FIG. 1) urging the operator to replace the container. During replacement, dialysis therapy is stopped and the operator manually disconnects the connector 19b/19c from empty container 3b/3c and attaches a full container 3b/3c. The control device 30 is then operated to resume dialysis therapy.

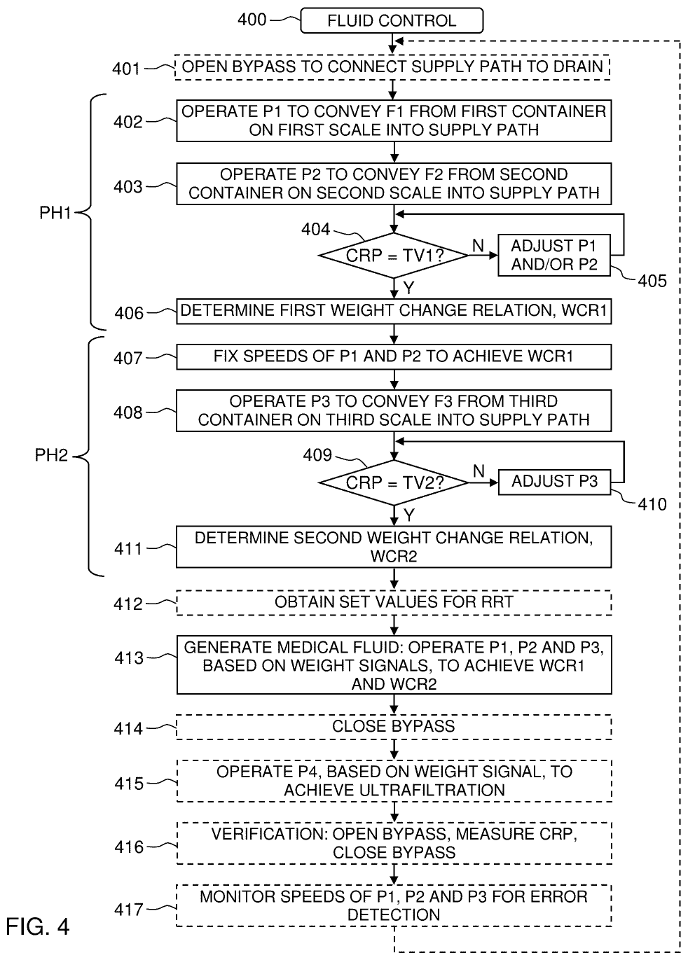

FIG. 4 is a flowchart of an example method 400 of controlling the fluid flows in the system 1. The method 400 may be executed by the control device 30. The method 400 comprises two preparatory phases PH1, PH2, which are performed before dialysis therapy and aim at deriving the above-mentioned “mixing control data” (MCD) by use of the CRP sensor 22. The MCD is then used by the control device 30 to control the pumps P1-P3 to generate the dialysis fluid during dialysis therapy. In FIG. 4, optional steps are indicated by dashed lines. While some of these optional steps are performed in the system of FIG. 1, they may be omitted in alternative systems, as will be discussed further below.

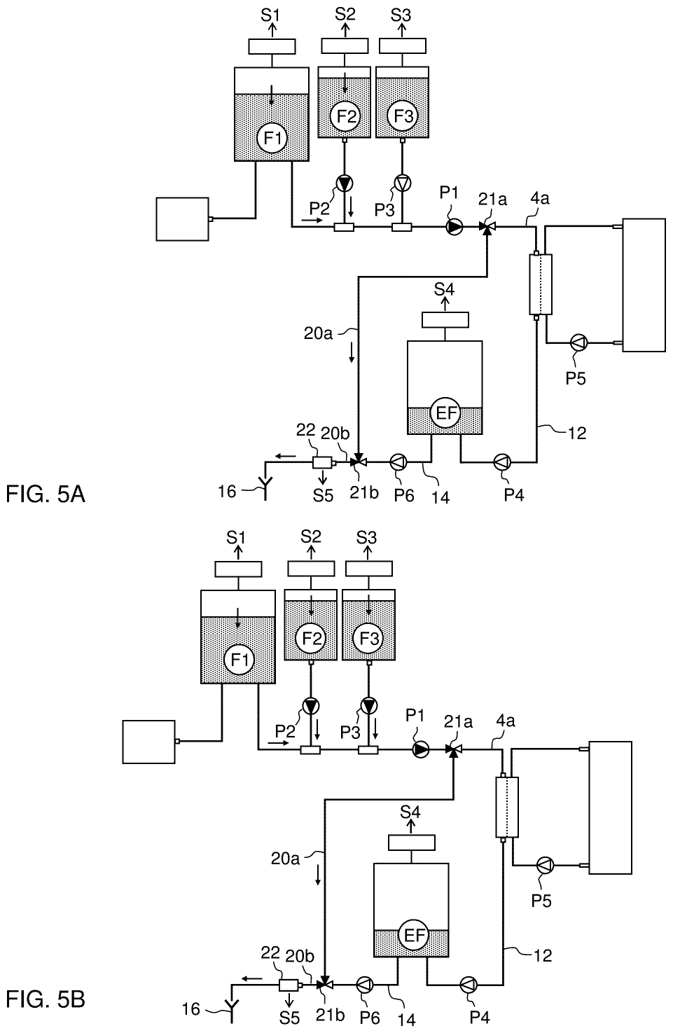

When PH1 is to be performed in the system of FIG. 1, the valve arrangement is set in its second state (step 401), as shown in FIG. 5A. Thereby, the first fluid line 4a (“supply path”) is closed, the bypass path is opened, and a passage from the supply path into the bypass path is opened. As seen in FIG. 5A, fluid is directed from line 4a via lines 20a, 20b to drain 16, while passing the CRP sensor 22. PH1 comprises steps 402-406. In step 402, pump P1 is operated to convey water (fluid F1) from the first container 3a into line 4a and lines 20a, 20b. In FIG. 5A, it is presumed that the pumps P2, P3 are occluding and thereby inherently block lines 4b, 4c when not activated. If necessary, controllable on/off valves (not shown) may be arranged along fluid lines 4b, 4c to selectively block fluid flows. In step 403, pump P2 is operated to convey A concentrate (fluid F2) from the second container 3b into line 4a and lines 20a, 20b. Pump P2 may be started at the same time as, or after, pump P1. FIG. 5A shows the system 1 when both pumps P1, P2 are operative during PH1. At this time, a mixture of F1 and F2 passes through the CRP sensor 22. In step 404, the signal S5 from the CRP sensor 22 is evaluated for determination of a current CRP value, and the current CRP value is compared to a first target CRP value, TV1, which defines a required property of the mixture of F1 and F2 as included in the dialysis fluid to be generated. TV1 may be predefined and stored in internal memory 32 of the control device 30 or be entered by an operator via the input device 34 (cf. FIG. 1). Alternatively, a selected mixing ratio between F1 and F2 may be entered by the operator and converted by the control device 30 into TV1. If the current CRP value is found to deviate from TV1, the speed of pump P1 and/or pump P2 is adjusted in step 405. Steps 404-405 are repeated until the current CRP value matches TV1. Steps 404-405 thus define a tuning procedure. In some embodiments, it may be desirable for the fluid flow in the supply path to be fixed during PH1. In the system shown in FIG. 5A, this may be simply achieved by only adjusting pump P2 in step 405. When the current CRP value matches TV1, a first weight change relation, WCR1, is determined in step 406 by use of signals S1, S2. WCR1 is part of the MCD and defines a relationship (\dot{W}1:\dot{W}2) between the weight change per unit time (\dot{W}1) for container 3a and a weight change per unit time (\dot{W}2) for container 3a. The weight change per unit time may be determined by operating any conventional differentiation algorithm on the respective signal S1, S2.

The method 400 then proceeds to perform PH2, which comprises steps 407-411. In step 407, pumps P1, P2 are operated to fulfill WCR1 according to the signals S1, S2, and the speeds of pumps P1, P2 are then fixed. In step 408, pump P3 is operated to convey B concentrate (fluid F3) from the third container 3c into line 4a and lines 20a, 20b. FIG. 5B shows the system 1 when pumps P1, P2, P3 are operative during PH2. At this time, a mixture of F1, F2 and F3 passes through the CRP sensor 22. In step 409, signal S5 from the CRP sensor 22 is evaluated for determination of a current CRP value, and the current CRP value is compared to a second target CRP value, TV2, which defines a required property of the dialysis fluid to be generated. TV2 may be obtained similar to TV1. If the current CRP value is found to deviate from TV2, the speed of pump P3 is adjusted in step 410. Steps 409-410 define a tuning procedure in correspondence with steps 404-405. Thus, steps 409-410 are repeated until the current CRP value matches TV2. When the current CRP value matches TV2, a second weight change relation, WCR2, is determined in step 411 by use of signals S1, S2, S3. WCR2 is part of the MCD and defines, depending on the definition of TV2, a relationship between the weight change per unit time (\dot{W}3) for container 3c and a weight change per unit time for container 3a and/or container 3b. Thus, the relationship may be given as \dot{W}3: \dot{W}1, \dot{W}3: \dot{W}2, or \dot{W}3: (\dot{W}1+\dot{W}2).

After completion of PH2, set values for the dialysis therapy to be performed are obtained in accordance with conventional practice (step 412). The set values may, for example, define the flow rate of fresh dialysis fluid to the dialyzer 7 (“main flow rate”), UFR, UF, blood flow rate through the dialyzer 7, etc. The set values may be retrieved from internal memory 32 or entered by an operator via the input device 34 (cf. FIG. 1).

In step 413, the system 1 is operated in accordance with the set values to generate the dialysis fluid. Step 413 may be performed directly after PH2 or at a later time. In step 413, pumps P1, P2, P3 are controlled based on signals S1, S2, S3 so as to achieve WCR1 and WCR2. In other words, pumps P1, P2, P3 are operated at speeds that result in weight changes of the containers 3a, 3b, 3c that fulfil WCR1 and WCR2. When WCR1 and WCR2 are fulfilled, the dialysis fluid is being generated. Step 413 may also comprise controlling pump P1 to supply the dialysis fluid at a target flow rate in given by the set values.

While step 413 is performed to generate the dialysis fluid, the valve arrangement is set in its first state (step 414). Thereby, line 4a (“supply path”) is opened, and the passage from line 4a into the bypass path is closed. In step 415, pump P4 may be operated based on signal S4 in relation to signals S1-S3 to achieve an UFR in accordance with the set values. Although not indicated in FIG. 4, pump P5 is operated to generate a blood flow rate in accordance with the set values. The system 1 is thereby operated to perform dialysis therapy as shown in FIG. 3A.

It may be noted that the system 1 is considered, by definition, to generate dialysis fluid whenever it is operated in accordance with the mixing control data, MCD. In the example of FIG. 4, the dialysis fluid is generated by mixing three fluids, and the MCD comprises WCR1 and WCR2. Thus, in this example, whenever the system 1 is operated to achieve WCR1 and WCR2, dialysis fluid is generated by the system 1. At all other times, the system is considered to generate a “mixture” of fluids, for example during the tuning procedures of PH1 and PH2.

In some embodiments, the main flow rate is changed during dialysis therapy, for example in accordance with a schedule included in the set values or by intervention by an operator via the input device 34 (FIG. 1) during on-going therapy. This may be achieved by jointly modifying the pumping speeds of pumps P1, P2, P3, based on signals S1-S3, so that WCR1 and WCR2 are fulfilled. In some embodiments, the valve arrangement may be intermittently set in its second state to open the bypass path when the main flow rate is to be changed, to avoid that a mixture of F1, F2, F3 that does not meet the specification of dialysis fluid is conveyed to the dialyzer 7 while pumps P1-P3 are adjusted.

As shown in FIG. 4, the method 400 may comprise a verification step 416, in which the bypass path is intermittently opened during on-going dialysis therapy, while pumps P1, P2, P3 are operating, to convey the dialysis fluid through the CRP sensor 22. Thereby, dialysis therapy is inherently interrupted. In step 416, a CRP value is determined based on signal S5 and compared to TV2. If the CRP value matches TV2, the bypass path is closed and dialysis therapy is thereby resumed. If a sufficient deviation is detected, the method 400 may initiate an execution of PH1 and PH2 to determine updated WCR1, WCR2. Alternatively, the method 400 may terminate dialysis therapy and/or generate an alert for the operator on the UI device (35 in FIG. 1).

The verification step 416 may be performed at regular intervals during therapy, or whenever a set value is changed during on-going therapy, or whenever a new container 3b, 3c of liquid concentrate has been installed. When a new container 3b, 3c has been installed, the method 400 may alternatively directly proceed to perform PH1 and PH2, without performing the verification step 416.

Alternatively or additionally, the verification step 416 may be performed whenever the speed of any one of the pumps P1-P3 is found to deviate from an operative speed range. The operative speed range may be defined as a range of allowable speed values for the respective pump, or as an allowable change of the speed of respective the pump, for example in relation to the initial speed of the respective pump when step 413 is started, i.e. when the dialysis fluid is first generated at the target flow rate.

Whenever the bypass path is opened during on-going therapy, the control device 30 may be configured to estimate the amount of dialysis fluid that is conveyed into the bypass path instead of being conveyed to the dialyzer 7, based on the signals S1-S3. The estimation may be given as a sum of the integrated weight decreases in the signals S1-S3 for the time period during which the bypass path is opened. This estimated amount may be deducted when the control device 30 calculates the accumulated amount of ultrafiltrate (UF) for the dialysis therapy.

As shown in FIG. 4, the method 400 may comprise a protective step 417, in which the speeds of the pumps P1-P3 are evaluated in relation to a set of speed limits for error detection. Errors in the system 1, for example a kinking of a fluid line or a mechanical failure of a pump, may cause the speed of a pump to change significantly. The set of speed limits defines the maximum and/or minimum speed limit for the respective pump. If the current speed of a pump is found to be below its minimum speed limit or above its maximum speed limit, step 417 may cause the system 1 to be shut down and/or cause an alarm to be generated via the UI device 35.

Alternative implementations of PH2 are possible. For example, only one of the pumps P1 and P2 may be active in step 407. This means that steps 407-408 result in a mixture of F1 and F3, or F2 and F3, which is conveyed through the CRP sensor 22 during the tuning procedure of steps 409-410. The resulting WCR2 defines the relationship \dot{W}1: \dot{W}3 or \dot{W}2: \dot{W}3.

The order in which the fluids F1-F3 are mixed in PH1 and PH2 may be selected in view of the required or available measurement range (calibration range) of the CRP sensor 22. The measurement range denotes the range of CRP values that are measured with sufficient accuracy by the sensor 22. Cheap CRP sensors 22 may have an available measurement range that is quite narrow. To minimize the required measurement range, the order of fluids may be selected to minimize the difference between TV1 and TV2. For example, if conductivity is measured by the CRP sensor 22, the smallest measurement range is obtained if PH1 is performed for a mixture of water and A concentrate (F1, F2), and PH2 is performed for a mixture of water, A concentrate and B concentrate (F1, F2, F3), i.e. as shown in FIG. 4.

As indicated by a dashed arrow in FIG. 4, the method 400 may return to the preparatory phases PH1, PH2 at any time, to thereby make a new determination of the MCD. As noted above, such a new determination may be triggered by the verification step 416. In another example, the method 400 may return to PH1 and PH2 when the composition of the medical fluid is to be changed, for example based on user input or in accordance with a predefined schedule. The change in composition is given by a change in TV1 and/or TV2 (cf. steps 404, 409).

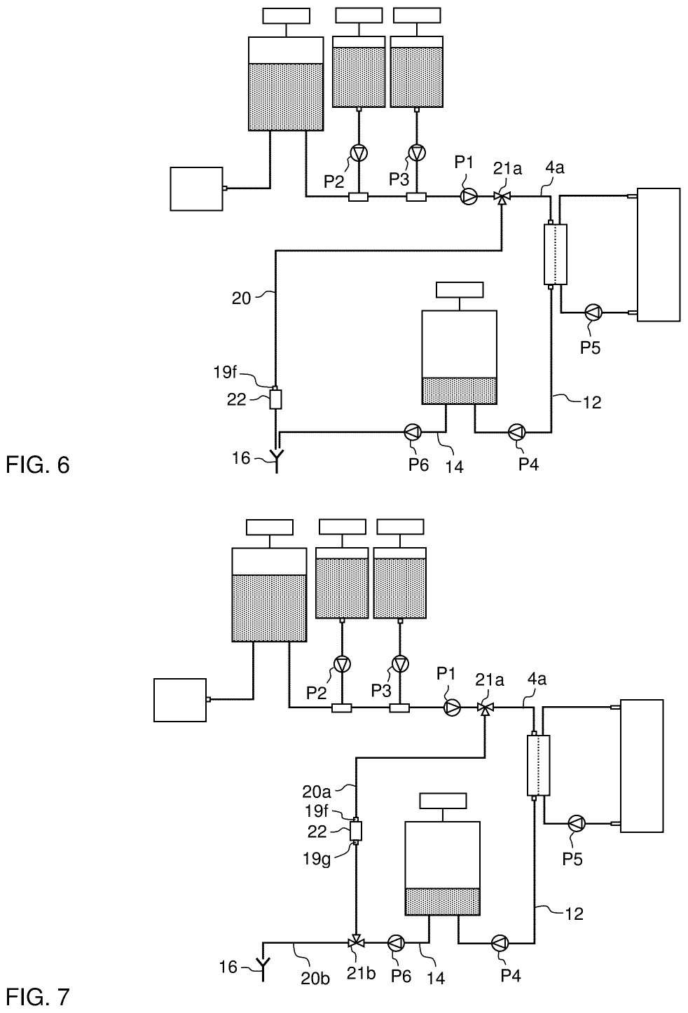

FIGS. 6-7 show alternative installations of the CRP sensor 22 in a bypass path in systems for dialysis therapy. The system in FIG. 6 differs from the system in FIG. 1 in that the bypass path is defined by a bypass line 20 that extends from line 4a to drain 16. The CRP sensor 22 is arranged in the bypass line 20, and the valve device 21b is omitted since it has no function here. As shown, the CRP sensor 22 may be releasably connected to the connector 19f, which is included in the disposable part (cf. FIG. 2A). The system in FIG. 7 differs from the system in FIG. 1 in that the CRP sensor 22 is located in the bypass line 20a. As shown, the CRP sensor 22 may be releasably connected to connectors 19f, 19g, which are included in the disposable part. One technical advantage of the systems in FIGS. 6-7 over the system in FIG. 1 is that the CRP sensor 22 is not exposed to effluent. This may further prolong the operative life of the CRP sensor 22 and/or reduce the need for rinsing, disinfection or other maintenance. On the other hand, when the CRP sensor 22 is exposed to effluent, it is possible to process signal S5, during a dedicated operating sequence for the system, to evaluate the efficacy of the dialysis therapy, for example in accordance with U.S. Pat. No. 5,024,756 or U.S. Pat. No. 6,217,539.

In the systems shown herein, it may be desirable to ensure a complete mixing of the fluids in the supply path (line 4a) upstream of its connection to the bypass path. Thereby, it is ensured that the fluid mixture that arrives at the CRP sensor 22 is homogeneous irrespective of how far downstream the bypass path the CRP sensor 22 is located.

As noted above, the control device 30 may be configured to control the speeds of the pumps P1-P3 based on the signals S1-S3 from the scales 2a-2c. To this end, the control device 30 may implement any conventional feedback control, including but not limited to P, PI or PID control. Should the time constant of the scales 2a-2c be large, causing the scale response to a weight change to be slow, the dynamic performance may be improved by so-called cascade control, which is well-known to the skilled person.

It should be understood that the dialysis fluid may be generated by mixing any number of fluids. Thus, the systems shown herein may comprise one or more additional containers of liquid concentrate arranged on a respective scale, and the method in FIG. 4 may be expanded with one or more additional preparatory phases for determination of weight change ratio. Likewise, the dialysis fluid may be generated by mixing only two fluids, which means that PH2 is omitted in the method 400, and that step 413 is modified to only operate pumps P1 and P2 to achieve WCR1 given by PH1.

It may also be noted that the amount of effluent conveyed from the dialyzer 7 need not be monitored by use of scale 2d. Instead, a flow meter or volumetric pumping may be used to monitor the amount of effluent. Thus, instead of collecting the effluent in the container 3d, the effluent may be directed directly to drain 16 or into a reservoir whose weight is not monitored.

In further variants of the system 1 in FIG. 1, the bypass path is omitted and thus also steps 401, 414 and 417 in the method of FIG. 4. Instead of being pumped into the bypass path during PH1 and PH2, the mixture may be pumped along the supply path (line 4a) to and through the dialyzer 7, if deemed not to be harmful to the patient. After passing the dialyzer 7, the mixture may be collected in the container 3d, or diverted from the effluent line 12 into a dedicated drain line that extends to drain 16. In such variants, the CRP sensor 22 would be located in the effluent line 12 or in the dedicated line. In another variant, line 4a is disconnected from the dialyzer 7 during PH1 and PH2 and instead connected to the CRP sensor 22. In the example of FIG. 1, connector 19d would be temporarily released from the dialyzer 7 and attached to the CRP sensor 22 instead of connector 19f.

The method of FIG. 4 is not limited to HD but may be applied in any system for RRT, including any modality of extracorporeal blood therapy and PD. FIG. 12 shows a system 1′ for generation of medical fluid for any type of RRT. The system 1′ need not be part of or connected to a system for dialysis therapy. The illustrated system 1′ is configured to generate the medical fluid by mixing two fluids F1, F2, but may be extended to admix further fluids if needed. Components in FIGS. 1 and 12 are identical insofar they are assigned the same reference numerals. The description will not be repeated for such components. In FIG. 12, the outlet of the first fluid line 4a (supply path) comprises a connector 19d, which is coupled to a connector 40a of a receiving device 40. The system also comprises a control device (not shown), which is configured to execute at least PH1 and step 413 of the method 400 in FIG. 4. Since the system in FIG. 12 comprises a bypass path, represented by the bypass line 20, the control device may also execute steps 401 and 414, as well as the verification step 416. The receiving device 40 is arranged to receive the medical fluid that is generated by execution of step 413. In some embodiments, the medical fluid is a dialysis fluid for use in extracorporeal blood therapy, such as HD or HDF, and the receiving device 40 comprises the dialyzer 7 (FIG. 1). In some embodiments, the medical fluid is a replacement fluid for use in HF or HDF, and the receiving device 40 comprises an infusion port on the withdrawal line 8a and/or on the return line 8b (FIG. 1). In some embodiments, the medical fluid is a dialysis fluid for use in PD, and the receiving device 40 comprises a disposable fluid circuit, which is attached to a PD cycler. It is also conceivable that the receiving device 40 corresponds to the peritoneal cavity as such. In some embodiments, which are applicable to all types of RRT, the receiving device 40 is a reservoir for collecting the medical fluid for subsequent use in RRT. In such embodiments, the medical fluid is not generated on-demand. The reservoir may or may not be connected to or part of a system for dialysis therapy.

In some embodiments, the system 1′ is included in a separate fluid generation apparatus which is arranged to supply medical fluid to a dialysis machine.

The system 1′ in FIG. 12 may be composed of a disposable part and a machine part, like the system 1 in FIG. 1. While distinct technical advantages are attained by confining the generation of the medical fluid to a disposable part, it is also possible to implement the systems 1, 1′ to generate the medical fluid within a permanent structure.

In some embodiments, some or all of the fluid lines in the disposable part are configured as passageways in a unitary cassette. The pumps and/or the valve arrangement may or may not be integrated in the cassette. If integrated, the pumps may, for example, be implemented as membrane pumps.

In further alternatives, the containers 3b, 3c may be refillable, by being fluidly connected to a respective source of liquid concentrate.

In the systems 1, 1′ shown in the drawings, the pump P1 is located in or on the first fluid line 4a downstream of junction 6′ (and junction 6″, if present). Thereby, the first pump P1 not only draws fluid F1 from container 3a but also defines the flow rate of the medical fluid (“main flow rate”). This gives the technical advantage of simplifying adjustment of the main flow rate. However, in some embodiments, the pump P1 may instead be located upstream of junction 6′.

In some embodiments, not shown, the system 1, 1′ further comprises one or more sterilizing grade filters, for example in the first fluid line 4a downstream of junction 6′ (and/or junction 6″, if present). It may be preferable to arrange the filter(s) close to the outlet for the medical fluid, for example between the valve device 21a and the connector 19d. The filter(s) may be configured to ensure that the medical fluid meets standards for ultrapure dialysis fluid or standards for replacement fluid in terms of viable bacteria (sterility) and endotoxins. Such filters are well-known in the art.

For reasons described hereinabove, the CRP sensor 22 may have a long operative life. In particular, the operative life of the CRP sensor 22 may exceed the operative life of the disposable part 1a (FIG. 2A). The CRP sensor 22 may thus be seen to be “re-usable”. To enable re-use, the disposable part is configured to be releasably attached to the CRP sensor 22. In the illustrated examples, the disposable part comprises one or more terminal connectors for establishing releasable fluid connection to the CRP sensor 22 (cf. 19f in FIGS. 1, 6, 7 and 12, and 19g in FIG. 7).

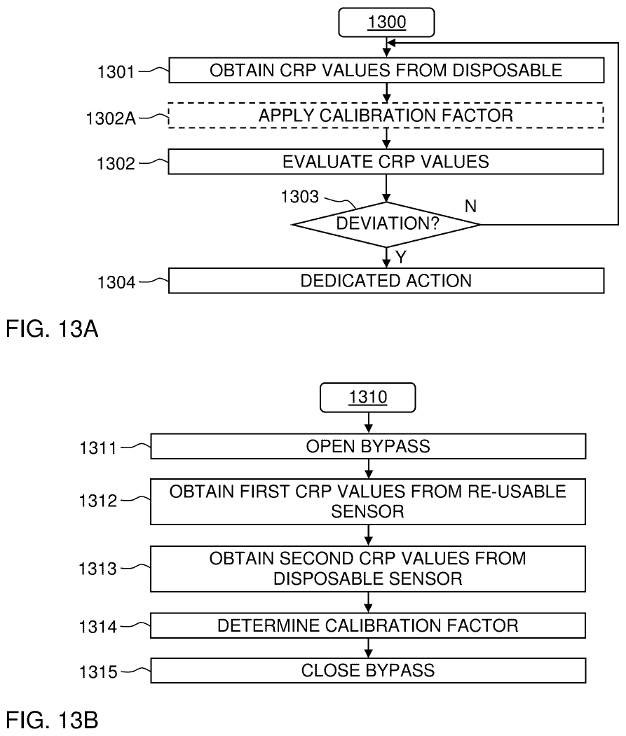

In some embodiments, the system includes an auxiliary CRP sensor in addition to the re-usable CRP sensor 22. In some embodiments, the auxiliary CRP sensor is a disposable component. In FIG. 12, a disposable CRP sensor 122 is arranged in line 4a upstream of the valve device 21a. The disposable sensor 122 may be a component of a disposable part, which may be configured to define fluid paths of the system by analogy with the disposable part 1a as described with reference to FIG. 2A. The disposable sensor 122 may be discarded together with the disposable part. The disposable sensor 122 is a low-end sensor device. While being relatively cheap, such a sensor device generally has poor performance in terms of accuracy and/or precision, which makes it unsuitable for use in determining the MCD. The purpose of the disposable sensor 122 is instead to detect changes in the composition of the medical fluid while it is being generated by step 413. Such changes may be harmful to the patient and may be caused by errors in the concentrate or water supplies, for example by an operator manipulating the content of a concentrate container. Thus, the control device may be configured to perform a safety procedure, while the medical fluid is being generated. The safety procedure presumes that the signal drift of the disposable sensor 122 is small during its deployment. An example safety procedure 1300 is shown in FIG. 13A. In the safety procedure, CRP values are obtained (step 1301) from the disposable sensor 122 and evaluated (step 1302) for detection of a deviation, which is representative of a change in composition. Upon detecting such a deviation (step 1303), the control device takes dedicated action (step 1304). The dedicated action may comprise, for example depending on the magnitude of the deviation, performing the verification step 416, shutting down the system 1′, generating an alarm, etc. To improve the performance of the disposable sensor 122, the control device may also perform a calibration procedure, for example while the medical fluid is being generated in accordance with step 413. An example calibration procedure 1310 is shown in FIG. 13B. In the calibration procedure 1310, the control device opens the bypass path, if not already open (step 1311). Then, the control device obtains (1312) a first set of CRP values from signal S5 of sensor 22 and a second set of CRP values from signal S5′ of disposable sensor 122 (step 1313). Steps 1312-1313 presume that the first and second sets of CRP values represent the same fluid, for example the medical fluid or any intermediate mixture (see below). The first and second sets of CRP values may but need not be obtained concurrently. The control device processes (step 1314) the first and second sets of CRP values to determine a calibration factor for the disposable sensor 122, under the assumption that the first set of CRP values are correct. The calibration factor is given by one or more parameter values of a conversion function that converts a CRP value measured by the disposable sensor 122 to a corresponding CRP value measured by the sensor 22. For example, the calibration factor may comprise an offset value (positive or negative) to be added to CRP values from the disposable sensor 122. The control device terminates the calibration procedure 1310 by storing the calibration factor in memory and closing the bypass path to start sending the medical fluid to the receiving device 40.

Steps 1312-1313 may be at least partly performed during the tuning procedure. In the example of FIG. 4, the first set of CRP values may comprise one or more CRP values measured at the end of or after preparatory phase PH2, i.e. when the current CRP values match the target value TV2 (cf. step 409). In the example of FIG. 4, step 1312 may also be performed at the end of or after preparatory phase PH1, when the current CRP values match the target value TV1 (cf. step 404). Thus, if the medical fluid is generated by mixing more than two constituent fluids, steps 1312-1313 may be performed to not only obtain CRP values for the medical fluid, but also CRP values for one or more intermediate mixtures generated during the tuning procedure. The use of the intermediate mixture(s) expands the range of the CRP values in the first and second sets. The accuracy of the calibration factor may be improved by accounting for the wider range of CRP values around the target value of the medical fluid. For example, in step 1314, the relation between the first and second CRP values within the range may be fitted to a line that represents a match between the first and second CRP values, for example by linear least squares. In this fitting, the CRP values may or may not be weighted, for example to relatively increase the impact of CRP values measured for the medical fluid compared to CRP values measured for the intermediate mixture(s). The fitting results in the above-mentioned conversion function, and thus also in the calibration factor. Further, since the conversion function is determined for a range of CRP values, the conversion function also defines the calibration factor to be applied in the safety procedure 1300 if the target value of the medical fluid (TV2) is changed within the range, which will happen if the composition of the medical fluid is changed.

As shown by step 1302A in FIG. 13A, the control device may be configured to retrieve and apply the calibration factor during the safety procedure, to correct CRP values from the disposable sensor 122 before evaluating the thus-corrected CRP values for detection of the deviation. The calibration procedure 1310 is facilitated by the disposable sensor 122 being located in the main supply path, so that it is exposed to the medical fluid, and upstream of the diversion from the main fluid path into the bypass path (control valve 21a), so that the sensors 22, 122 are concurrently exposed to the medical fluid. However, it is conceivable that the disposable sensor 122 is instead located downstream of the control valve 21a, and that the control device obtains (in step 1312) the first set of CRP values while the bypass path is opened and the second set of CRP values while the bypass path is closed.

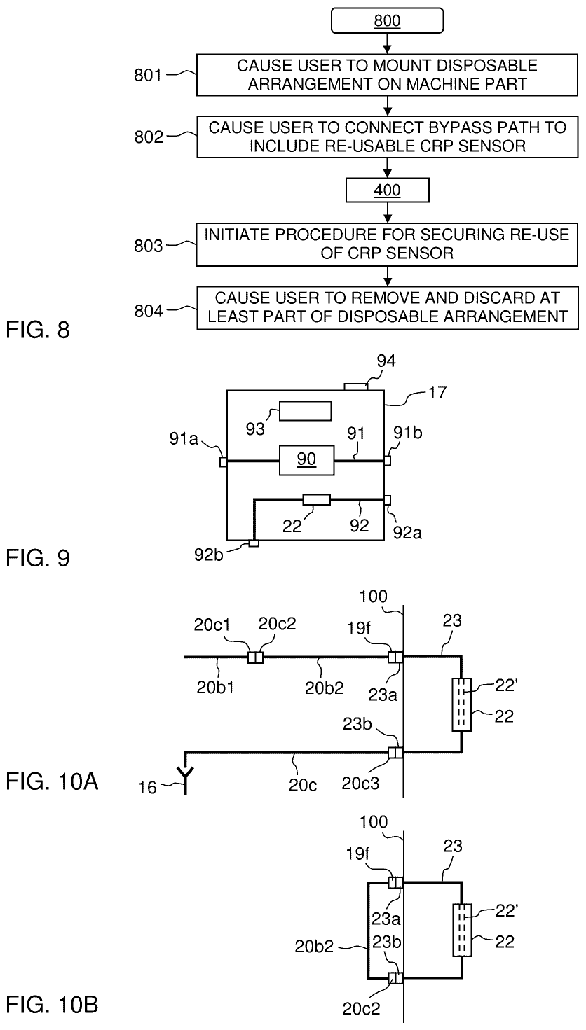

FIG. 8 is a flow chart of an example method 800 of configuring and operating a system to generate medical fluid. The method 800 may be performed by a control device in any system described herein. In step 801, a user (operator) is caused to mount the disposable part on the machine part, in proper engagement with scales, pumps, etc. The machine part may be a dialysis machine or any other machine equipped with required pumps, scales, etc. In step 802, the user is caused to connect the disposable part to the re-usable CRP sensor 22 so that the re-usable CRP sensor 22 in included in the bypass path. Then, the method 400 of FIG. 4 is executed to generate the medical fluid. At a certain time point, the method 400 is terminated and step 803 is performed to initiate a procedure for securing re-use of the CRP sensor 22. Examples of step 803 are described below. When step 803 is completed, step 804 is performed to cause the user to remove the disposable part from the machine part and discard the disposable part. It is conceivable that a subset of the disposable part is re-usable and is left on the machine part in step 804. For example, if the containers 3b, 3c are included in the disposable part, the containers 3b, 3c may be left on the scales 2b, 2c. Steps 801-804 may comprise providing user instructions on the UI device 35 (FIG. 1) to cause the user to perform one or more actions. Thus, in the context of the present disclosure, causing a user to perform an action is equivalent to outputting an instruction to the user to perform the action.

Step 803 may differ depending on location and degree of integration of the CRP sensor 22 within the system.

In some embodiments, the CRP sensor 22 is integrated in the water supply device 17. An example of such a supply device 17 is shown in FIG. 9. The operation of the supply device 17 is controlled by an internal controller (not shown). The device 17 comprises a water processing unit 90, which is operable to process incoming water to produce purified water for use in the medical fluid. The water processing unit 90 may be configured to purify the incoming water by any available technique, such as sediment filter, carbon filter, resin bed, ultrafiltration (UF), reverse osmosis (RO), nanofiltration, electrodeionization (EDI), or capacitive deionization (CDI). The device 17 comprises a water supply line 91 that extends from an inlet connector 91a to an outlet connector 91b. In operation, a source of tap water may be connected to the inlet connector 91a, and the terminal connector 19a of the disposable part (FIGS. 1 and 12) may be connected to the outlet connector 91b. The device 17 further comprises an auxiliary fluid line 92, which extends from an inlet connector 92a to an outlet connector 92b via the sensor 22. In operation, the terminal connector 19f of the disposable part (FIGS. 1, 6, 7 and 12) may be connected to the inlet connector 92a, and a fluid line (not shown) extending to the drain 16 may be connected to the outlet connector 92b. The device 17 further comprises a cleaning unit 93, which is operable to perform a cleaning operation on the sensor 22 and optionally the water processing unit 90. The cleaning operation may include rinsing and/or disinfecting. As used herein, rinsing involves flushing the CRP sensor by a fluid to remove deposits, particles, etc. The fluid may be any liquid, including water, and may or may not comprise a cleaning agent, such as a detergent, a descaling agent, etc. As used herein, disinfecting refers to a process of preventing growth of microorganisms in the CRP sensor and may involve heat treatment, flushing with a disinfectant or sterilant, etc. It is to be understood that not only the CRP sensor 22 is treated in the cleaning operation but also connecting fluid paths. The device 17 further comprises an I/O unit 94, which is operable to output the signal S5 and optionally to receive control signals. In operation, the I/O unit 94 is connected to the control device 30, by wire or wirelessly. In one implementation of step 803, the control device 30 transmits a control signal to the supply device 17 to initiate the cleaning operation. In another implementation of step 803, the control device 30 causes the user, via the UI device 35 (FIG. 1), to initiate the cleaning operation, for example by pushing a dedicated button (not shown) on the device 17.

Since the CRP sensor 22 is located in the bypass path, growth of microorganisms in the CRP sensor 22 is less of a risk factor compared to when the CRP sensor 12 is arranged in the main supply path (line 4a). The need for disinfection is thereby reduced, or even eliminated. Nevertheless, since the CRP sensor 22 is re-used in plural sessions, periodic cleaning may be performed to ensure proper functioning of the CRP sensor 22 over time. For example, rinsing may be performed to mitigate fouling, for example by scaling, deposits, etc. Cleaning to prevent growth of microorganisms in the CRP sensor 22 may be relevant whenever there is a risk of microorganisms moving along the bypass path into the main supply path. This risk may, for example, depend on the distance from the CRP sensor 22 to the main supply path and/or whether the bypass path is intermittently opened while the medical fluid is being generated (cf. verification step 416). It is realized that the cleaning operation may involve rinsing and/or disinfection and that rinsing and disinfection may be performed at different time intervals. Further, unlike the method 800 in FIG. 8, it is conceivable to perform the cleaning operation less frequently than after every session.

In some embodiments, the CRP sensor 22 is integrated in the machine part 1b (cf. FIG. 2B). If the machine part comprises an integrated cleaning unit for cleaning the sensor 22, the control device 30 may autonomously initiate the cleaning operation in step 803.

In some embodiments, the CRP sensor 22 is included in a separate device (not shown), which may or may not be attached to the machine part 1b or the supply device 17.

FIGS. 10A-10B show an example of a CRP sensor 22, which is arranged inside a device 100, which may be the machine part 1b, the supply device 17 or the above-mentioned separate device. Here, it is assumed that the device 100 lacks functionality for rinsing or disinfecting the sensor 22. The sensor 22 defines an internal channel 22′ and is configured to measure CRP values for fluid in the channel 22′. The device 100 defines an inlet port 23a and an outlet port 23b, which are in fluid communication with the channel 22′ on a flow path 23. Although not shown, the device 100 comprises an I/O unit for transfer of measurement data (cf. signal S5) to the control device 30. FIG. 10A shows a first connection state, which is attained in step 802 of FIG. 8. In the first connection state, the sensor 22 is arranged in the bypass path. In the illustrated example, the bypass path comprises first and second interconnectable line segments 20b1, 20b2, which jointly correspond to line 20b in FIG. 1, line 20 in FIG. 6 or FIG. 12, or line 20a in FIG. 7. Specifically, line segment 20b2 comprises terminal connector 19f, which is configured for connection to inlet port 23a, and terminal connector 20c2, which is configured for connection to terminal connector 20cl on line 20b1. Further, a line segment 20c is connected by terminal connector 20c3 to outlet port 23b and extends to drain 16. FIG. 10B shows a second connection state, which is attained in step 803 of FIG. 8. In the second connection state, the sensor 22 is encapsulated in a closed loop and is filled with a dedicated fluid, which is configured to prolong the operative life of the sensor 22. For example, the dedicated fluid may be a bacteriostatic fluid. A bacteriostatic fluid comprises a bacteriostatic agent, which is a biological or chemical agent that stops bacterial growth, or even kills bacteria (also known as bactericidal agent). Thus, in the second connection state, the sensor 22 kept in an environment that prevents bacterial growth. To attain the second connection state, step 803 may comprise operating the system 1, 1′ to convey the dedicated fluid from a supply into the bypass path and through the sensor 22. When the sensor channel 22′ contains the dedicated fluid, which may be detected from the signal S5, step 803 comprises instructing the user to disconnect line 20b1 from line 20b2, disconnect line 20c from port 23b, and attach terminal connector 20c2 to port 23b. By this manipulation, the dedicated fluid is retained within the sensor 22. The manipulation in FIGS. 10A-10B involve re-using a component of the disposable part to fluidly connect the ports 23a, 23b. When the method 800 is to be performed at a later time, at which the device 100 is in the second connection state, step 802 may comprise instructing the user to disconnect and discard line segment 20b2 before connecting a new disposable part to the device 100 in accordance with the first connection state (FIG. 10A).

The dedicated fluid may be distinct from the fluids F1, F2, F3 and may be conveyed into the bypass path from a separate source by a dedicated pump. However, in some embodiments, the dedicated fluid comprises at least one of the liquid concentrates that are included in the medical fluid. For example, the A concentrate has bacteriostatic properties and may be used as dedicated fluid in step 803. With reference to FIG. 1, the valve arrangement 21a, 21b may be set in its second state, pump P3 may be stopped and pumps P1, P2 may be operated at identical speeds to convey A concentrate into the sensor 22. It is realized that the ability to use one of the liquid concentrates as dedicated fluid in step 803 greatly simplifies the structure of the system 1, 1′.

FIGS. 10A-10B only show one example of how the sensor 22 may be manipulated to retain the dedicated fluid within the sensor. For example, the ports 23a, 23b may be sealed in other ways to retain the dedicated fluid. Further, the technique of retaining a dedicated fluid within the sensor 22 in step 803 is also applicable if the sensor 22 is integrated within the machine part 1b or the supply device 17.

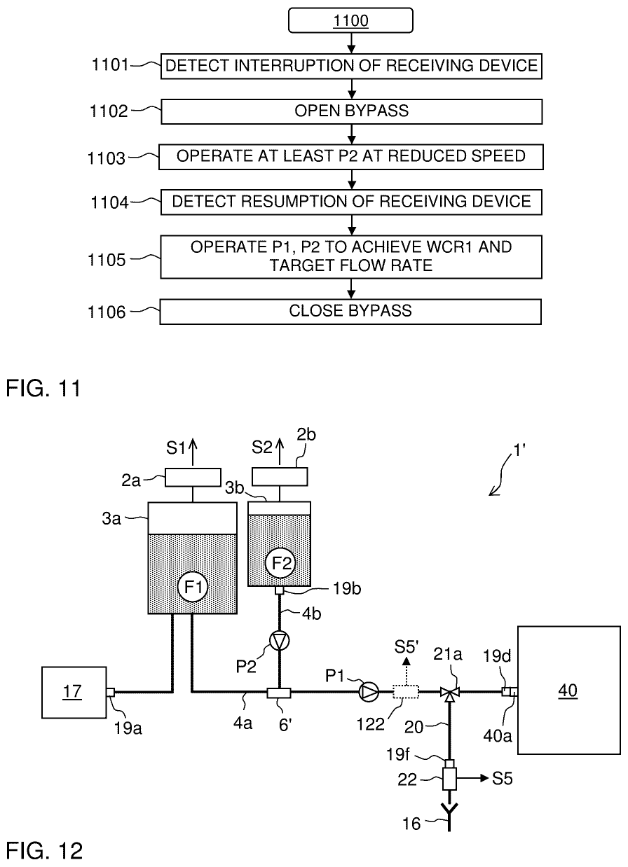

FIG. 11 is a flowchart of an example method 1100 which may be performed during generation of the medical fluid in the system 1, 1′. The method 1110 will be described with reference to the system 1′ in FIG. 12. When an interruption in the operation of the receiving device 40 is detected (step 1101), the valve device 21a is operated to open the bypass path to direct the medical fluid to drain 16 (step 1102). Here, it is presumed that the interruption prevents the device 40 from receiving the medical fluid. The control device of the system 1′ may detect the interruption based on an output signal from the receiving device 40 or from a sensor, for example a pressure sensor (not shown) in line 4a. In conjunction with step 1102, to minimize the waste of liquid concentrate F2, the speed of at least pump P2 is reduced but not stopped (step 1103). Thus, both pumps P1, P2 are still operative. When a resumed operation of the receiving device 40 is detected (step 1104), the speeds of pumps P1, P2 are controlled to meet the target flow rate and to achieve WCR1 (step 1105). In conjunction with step 1104, the valve device 21a is operated to close the bypass path to direct the medical fluid towards the receiving device 40 (step 1106). It is conceivable to implement step 1104 with a timeout function, which causes the system 1′ to be shut down if resumed operation is not detected within a predefined time from step 1101.

By the method 1100, pumps P1, P2 are kept operating during the interruption of the receiving device 40. If pumps P1, P2 were to be stopped, hysteresis effects may require the control device to re-initiate PH1 to update WCR1. It is conceivable that both pumps P1, P2 are operated to reduce their speeds in step 1103. Further, when operated at the reduced speeds, pumps P1, P2 may be controlled to maintain WCR1, given by signals S1, S2. Thereby, medical fluid is still generated in step 1103, albeit at a reduced flow rate. This makes it possible for the control device to verify, from signal S5, that the CRP value meets TV1 throughout the interruption and will also further reduce the risk of hysteresis effects.

There are commercially available concentrates that may be used in the fluid generation system 20 as described herein.

In some embodiments, dialysis fluid for treatment of CKD patients by hemodialysis, hemofiltration or hemodiafiltration is generated by mixing a single concentrate with water at a dilution ration of 10-50 by volume. In a non-limiting example, the single concentrate comprises lactate, sodium, potassium, calcium, magnesium, glucose and chloride. Such a concentrate is, for example, commercially available for the PureFlow SL system from NxStage. Alternatively, the dialysis fluid may be generated by mixing two concentrates with water. For example, a bicarbonate concentrate and an acid concentrate may be mixed with water at a dilution ratio of 10-50. Such concentrates are commercially available and well-known in the art. In a non-limiting example, the bicarbonate concentrate comprises bicarbonate, and the acid concentrate comprises sodium, potassium, calcium, magnesium, glucose, acetate and chloride. In some acid concentrates, acetate is replaced or supplemented by another acid, for example citric acid or hydrochloric acid.

In some embodiments, dialysis fluid for CRRT treatment of AKI patients is generated by mixing at least one concentrate with water. In a non-limiting example, such a dialysis fluid comprises bicarbonate, sodium, potassium, calcium, magnesium, phosphate, glucose, acetate and chloride. In one example, a base concentrate and an electrolyte concentrate may be mixed with water to form the dialysis fluid. For example, the base concentrate may be an alkaline bicarbonate solution, and the electrolyte concentrate may be an acidic glucose-based electrolyte solution.

In some embodiments, dialysis fluid for use in peritoneal dialysis (PD) is generated by mixing at least one concentrate with water. Example compositions of PD concentrates, to be mixed with water individually or in combination, are disclosed in US2018/0021501 and WO2017/193069, which are incorporated herein by reference.

While the subject of the present disclosure has been described in connection with what is presently considered to be the most practical embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

In the following, clauses are recited to summarize some aspects and embodiments as disclosed in the foregoing.
C1. A computer-implemented method of generating a medical fluid for renal replacement therapy, said method comprising: operating (402) a first pump (P1) to convey a first fluid (F1) from a first container (3a), which is arranged on a first scale (2a), into a supply path (4a) that extends to an outlet (19d) for the medical fluid; operating (403) a second pump (P2) to convey a second fluid (F2) from a second container (3b), which is arranged on a second scale (2b), into the supply path (4a), to generate a mixture of the first and second fluids (F1, F2) in the supply path (4a); measuring (404), by a sensor (22), a composition-related parameter of the mixture; adjusting (405) a pumping speed of at least one of the first and second pumps (P1, P2) until the sensor (22) measures a target value of the composition-related parameter; determining (406), based on first and second output signals (S1, S2) from the first and second scales (2a, 2b), a relation between a first weight change of the first scale (2a) and a second weight change of the second scale (2b) while the sensor (22) measures the target value; and generating (413) the medical fluid in the supply path (4a), said generating (413) comprising operating, based on the first and second output signals (S1, S2), the first and second pumps (P1, P2) to achieve said relation between the first and second weight changes.
C2. The method of C1, wherein the first and second pumps (P1, P2) are operated concurrently to convey the first and second fluids (F1, F2) into the supply path (4a).
C3. The method of C1 or C2, further comprising, before said measuring (404), operating (401) a valve arrangement (21a, 21b) to redirect the mixture from the supply path (4a) into a bypass path (20; 20a, 20b) which directs the mixture through the sensor (22).
C4. The method of C3, further comprising, when the medical fluid is generated in the supply path (4a), operating (414) the valve arrangement (21a, 21b) to direct the medical fluid along the supply path (4a) to the outlet (19d) for the medical fluid.
C5. The method of C3 or C4, wherein the medical fluid is generated in the supply path (4a) upstream of a connection point where the bypass path (20; 20a, 20b) is joined to the supply path (4a).
C6. The method of C4 or C5, wherein the outlet (19d) is connected to a receiving device (40), said method further comprising: detecting (1101), while the medical fluid is directed along the supply path (4a) to the outlet (19d), an interrupted operation of the receiving device (40); and, upon detecting the interrupted operation, operating (1102) the valve arrangement (21a, 21b) to close the supply path (4a) and open the bypass path (20; 20a, 20b) and reducing (1103) the pumping speed of at least one of the first and second pumps (P1, P2).
C7. The method of C6, wherein said reducing (1103) further comprises maintaining said relation between the first and second weight changes.
C8. The method of any one of C3-C7, further comprising, before said operating (402) the first pump (P1) and said operating (403) the second pump (P2), causing (802) the user to fluidly connect the bypass path (20; 20a, 20b) to the sensor (22).
C9. The method of claim C8, further comprising: initiating (803), after completion of the renal replacement therapy, a procedure for securing re-use of the sensor (22).
C10. The method of C9, wherein the procedure for securing re-use comprises at least one of: a) operating a fluid supply device (17; 1b), which comprises the sensor (22), to perform an operation of rinsing and/or disinfecting the sensor (22), or b) conveying a dedicated fluid through the bypass path (20; 20a, 20b) into the sensor (22), and causing the user to disconnect the bypass path (20; 20a, 20b) from the sensor (22) and manipulate the sensor (22) to retain the dedicated fluid within the sensor (22).
C11. The method of C10, wherein the dedicated fluid is bacteriostatic.
C12. The method of C10 or C11, wherein said conveying a dedicated fluid comprises: operating the second pump (P2) to convey the second fluid (F2) via the supply path (4a) and the bypass path (20; 20a, 20b) into the sensor (22), or operating a third pump (P3) to convey a third fluid (F3) via the supply path (4a) and the bypass path (20; 20a, 20b) into the sensor (22), said third fluid (F3) being included in the medical fluid together with the second fluid (F2).
C13. The method of any preceding clause, wherein the supply path (4a) is discarded after completion of the renal replacement therapy.
C14. The method of any preceding clause, wherein said generating (413) the medical fluid comprises: controlling the pumping speeds of the first and second pumps (P1, P2) to generate the medical fluid at a target flow rate, while maintaining said relation between the first and second weight changes.
C15. The method of C14, wherein the target flow rate matches a consumption rate of the medical fluid by said renal replacement therapy.
C16. The method of any preceding clause, wherein the first fluid (F1) is water and the second fluid (F2) is a liquid concentrate.
C17. The method of C16, wherein said adjusting (405) comprises: adjusting the pumping speed of the second pump (P2) until the sensor (22) measures the target value of the composition-related parameter.
C18. The method of any preceding clause, further comprising: monitoring (416, 417), while the medical fluid is generated in the supply path (4a), the pumping speeds of the first and second pumps (P1, P2) for detection of changes indicative of operational error.
C19. The method of any preceding clause, further comprising: activating (408) a third pump (P3) to convey a third fluid (F3) from a third container (3c), which is arranged on a third scale (2c), into the supply path (4a) while the first pump (P1) is operated to convey the first fluid (F1) into the supply path (4a) and/or the second pump (P2) is operated to convey the second fluid (F2) into the supply path (4a), to generate a further mixture in the supply path (4a); measuring (409), by the sensor (22), the composition-related parameter of the further mixture; adjusting (410) the pumping speed of the third pump (P3) until the sensor (22) measures a further target value of the composition-related parameter; and determining (411), based on a third output signal (S3) from the third scale (2c), a further relation between a third weight change of the third scale (2c) and at least one of the first and second weight changes while the sensor (22) measures the further target value, wherein said generating (414) comprises operating the third pump (P3), based on the third output signal (S3), to achieve said further relation between the third weight change and said at least one of the first and second weight changes.
C20. The method of C19, wherein, during said adjusting (410), the first and second pumps (P1, P2) are concurrently operated to convey the first and second fluids (F1, F2) into the supply path (4a), and the pumping speeds of the first and second pumps (P1, P2) are fixed to achieve said relation between the first and second weight changes.
C21. The method of any preceding clause, wherein the composition-related parameter represents conductivity, resistivity, or concentration of one or more solutes, or pH.
C22. The method of any preceding clause, wherein the medical fluid is a treatment fluid for use in extracorporeal blood therapy or peritoneal dialysis therapy.
C23. A control device, comprising circuitry (31, 32), which is configured to perform the method of any one of C1-C22, and a signal interface (33a), which is configured to output control signals (C1, C2) for the first and second pumps (P1, P2) and further configured to receive the first and second output signals (S1, S2) from the first and second scales (2a, 2b) and a sensor signal (S5) representative of the composition-related parameter from said sensor (22).
C24. A computer-readable medium comprising computer instructions which, when executed by a processor (31), cause the processor (31) to perform the method of any one of C1-C22.
C25. A system for generating a medical fluid for renal replacement therapy, said system comprising: a first scale (2a); a first container (3a) arranged on the first scale (2a); a second scale (2b); a second container (3b) arranged on the second scale (3b); a supply path (4a), which is configured to receive a first fluid (F1) from the first container (3a) and a second fluid (F2) from the second container (3b) and extends to an outlet (19d) for the medical fluid; a first pump (P1) arranged to convey the first fluid (F1) from the first container (3a) into the supply path (4a); a second pump (P2) arranged to convey the second fluid (F2) from the second container (3b) into the supply path (4a) to generate a mixture of the first and second fluids (F1, F2) in the supply path (4a); a sensor (22) configured to measure a composition-related parameter; and the control device (30) according to C23.
C26. The system of C25, wherein the sensor (22) is re-usable.
C27. The system of C25 or C26, further comprising a bypass path (20; 20a, 20b), which is fluidly connected to the supply path (4a) and extends to the sensor (22); and a valve arrangement (21a, 21b), which is operable to selectively direct the mixture from the supply path (4a) into the bypass path (20; 20a, 20b).
C28. The system of C27, further comprising an auxiliary sensor (122), which is configured to measure the composition-related parameter and is arranged in the supply path (4a), wherein the control device (30) is configured to perform a safety procedure (1300) while generating the medical fluid in the supply path (4a), wherein the safety procedure (1300) comprises: obtaining measurement values representative of the medical fluid from the auxiliary sensor (112), evaluating the measurement values for detection of a deviation, and performing a dedicated action upon detection of the deviation.
C29. The system of C28, wherein the auxiliary sensor (122) is a disposable component.
C30. The system of C28 or C29, wherein the control device (30) is further configured to perform a calibration procedure (1310) while generating the medical fluid in the supply path (4a), wherein the calibration procedure (1310) comprises: operating the valve arrangement (21a, 21b) to direct the medical fluid from the supply path (4a) into the bypass path (20; 20a, 20b), obtaining a first set of measurement values representative of the medical fluid from the sensor (22), obtaining a second set of measurement values representative of the medical fluid from the auxiliary sensor (122), and calculating a calibration factor based on the first and second sets of measurement values, wherein the control device (30) is configured to, in the safety procedure (1300), adjust the measurement values by the calibration factor before evaluating the measurement values.
C31. The system of any one of C27-C30, which comprises a machine part (1b) and a disposable arrangement (1a) releasably engaged with the machine part (1b), wherein the machine part (1b) comprises the first and second scales (2a, 2b), the first and second pumps (P1, P2), and the valve arrangement (21a, 21b), and wherein the disposable arrangement (1a) defines the supply path (4a) and the bypass path (20; 20a, 20b).
C32. The system of any one of C27-C31, wherein the sensor (22) is releasably connected to the bypass path (20; 20a, 20b).
C33. The system of any one of C27-C32, which is operable to convey a dedicated fluid through the bypass path (20; 20a, 20b) into the sensor (22), and to instruct a user to disconnect the bypass path (20; 20a, 20b) from the sensor (22) and manipulate the sensor (22) to retain the dedicated fluid within the sensor (22).
C34. The system of C33, wherein the dedicated fluid comprises the second fluid (F2) and/or a third fluid (F3), which is optionally included in the medical fluid, and wherein the dedicated fluid is bacteriostatic.
C35. The system of any one of C27-C34, wherein the sensor (22) is included in a fluid supply device (17), which is configured to supply the first fluid (F1) and is fluidly connected to the first container (3a).
C36. The system of C35, wherein the fluid supply device (17) is operable to perform a procedure of rinsing and/or disinfection of the sensor (22).
C37. The system of any one of C25-C36, wherein a connecting line (4b) extends from the second container (3a) to a junction (6′) on the supply path (4a) and the second pump (P2) is arranged in or on the second connecting line (4b) to convey the second fluid (F2) from the second container (3a) into the supply path (4a), and wherein the first pump (P1) is arranged in or on the supply path (4a) between the junction (6′) and the outlet (19d) for the medical fluid.
C38. A disposable arrangement for use in the system of any one of claims 27-38, said disposable arrangement comprising: the first container (3a); the supply path (4a); a connecting line (4b), which is in fluid communication with the supply path (4a) and extends to a first terminating fluid connector (19b), which is configured for connection to the second container (3b); and the bypass path (20; 20a, 20b), which extends from the supply path (4a) to a second terminating fluid connector (19f), which is configured for connection to an inlet connector (23a; 92a) in fluid communication with the sensor (22).

1. A computer-implemented method of generating a medical fluid for renal replacement therapy, the method comprising:
operating a first pump to convey a first fluid from a first container, which is arranged on a first scale, into a supply path that extends to an outlet for the medical fluid,
operating a second pump to convey a second fluid from a second container, which is arranged on a second scale, into the supply path, to generate a mixture of the first and second fluids in the supply path,
measuring, by a sensor, a composition-related parameter of the mixture,
adjusting a pumping speed of at least one of the first and second pumps until the sensor measures a target value of the composition-related parameter,
determining, based on first and second output signals from the first and second scales, a relation between a first weight change of the first scale and a second weight change of the second scale while the sensor measures the target value, and
generating the medical fluid in the supply path, the generating comprising operating, based on the first and second output signals, the first and second pumps to achieve the relation between the first and second weight changes.

2. The method of claim 1, further comprising, before the measuring, operating a valve arrangement to redirect the mixture from the supply path into a bypass path which directs the mixture through the sensor.

3. The method of claim 2, further comprising, when the medical fluid is generated in the supply path, operating the valve arrangement to direct the medical fluid along the supply path to the outlet for the medical fluid.

4. The method of claim 3, wherein the outlet is connected to a receiving device, the method further comprising: detecting, while the medical fluid is directed along the supply path to the outlet, an interrupted operation of the receiving device; and, upon detecting the interrupted operation, operating the valve arrangement to close the supply path and open the bypass path and reducing the pumping speed of at least one of the first and second pumps.

5. The method of claim 4, wherein the reducing further comprises maintaining the relation between the first and second weight changes.

6. The method of claim 2, further comprising, before the operating the first pump and the operating the second pump, causing a user to fluidly connect the bypass path to the sensor.

7. The method of claim 6, further comprising: initiating, after completion of the renal replacement therapy, a procedure for securing re-use of the sensor.

8. The method of claim 7, wherein the procedure for securing re-use comprises at least one of: a) operating a fluid supply device, which comprises the sensor, to perform an operation of rinsing and/or disinfecting the sensor, or b) conveying a dedicated fluid through the bypass path into the sensor, and causing the user to disconnect the bypass path from the sensor and manipulate the sensor to retain the dedicated fluid within the sensor.

9. The method of claim 8, wherein the dedicated fluid is bacteriostatic.

10. The method of claim 8, wherein the conveying the dedicated fluid comprises: operating the second pump to convey the second fluid via the supply path and the bypass path into the sensor, or operating a third pump to convey a third fluid via the supply path and the bypass path into the sensor, the third fluid being included in the medical fluid together with the second fluid.

11. The method of claim 1, further comprising: monitoring, while the medical fluid is generated in the supply path, the pumping speeds of the first and second pumps for detection of changes indicative of operational error.

12. The method of claim 1, further comprising:
activating a third pump to convey a third fluid from a third container, which is arranged on a third scale, into the supply path while the first pump is operated to convey the first fluid into the supply path and/or the second pump is operated to convey the second fluid into the supply path, to generate a further mixture in the supply path,
measuring, by the sensor, the composition-related parameter of the further mixture,
adjusting the pumping speed of the third pump until the sensor measures a further target value of the composition-related parameter, and
determining, based on a third output signal from the third scale, a further relation between a third weight change of the third scale and at least one of the first and second weight changes while the sensor measures the further target value,
wherein the generating comprises operating the third pump, based on the third output signal, to achieve the further relation between the third weight change and the at least one of the first and second weight changes.

13. The method of claim 12, wherein, during the adjusting, the first and second pumps are concurrently operated to convey the first and second fluids into the supply path, and the pumping speeds of the first and second pumps are fixed to achieve the relation between the first and second weight changes.

14. A control device, comprising circuitry, which is configured to perform the method of claim 1, and a signal interface, which is configured to output control signals for the first and second pumps and further configured to receive the first and second output signals from the first and second scales and a sensor signal representative of the composition-related parameter from said sensor.

15. A computer-readable medium comprising computer instructions which, when executed by a processor, cause the processor to perform a method for generating a medical fluid for renal replacement therapy, the method comprising:
operating a first pump to convey a first fluid from a first container, which is arranged on a first scale, into a supply path that extends to an outlet for the medical fluid,
operating a second pump to convey a second fluid from a second container, which is arranged on a second scale, into the supply path, to generate a mixture of the first and second fluids in the supply path,
measuring, by a sensor, a composition-related parameter of the mixture,
adjusting a pumping speed of at least one of the first and second pumps until the sensor measures a target value of the composition-related parameter,
determining, based on first and second output signals from the first and second scales, a relation between a first weight change of the first scale and a second weight change of the second scale while the sensor measures the target value, and
generating the medical fluid in the supply path, the generating comprising operating, based on the first and second output signals, the first and second pumps to achieve the relation between the first and second weight changes.

16. A system for generating a medical fluid for renal replacement therapy, said system comprising:
a first scale;
a first container arranged on the first scale;
a second scale;
a second container arranged on the second scale;
a supply path, which is configured to receive a first fluid from the first container and a second fluid from the second container and extends to an outlet for the medical fluid;
a first pump arranged to convey the first fluid from the first container into the supply path;
a second pump arranged to convey the second fluid from the second container into the supply path to generate a mixture of the first and second fluids in the supply path;
a sensor configured to measure a composition-related parameter; and
a control device comprising circuitry, which is configured to perform a method, wherein the method comprises:
operating the first pump to convey the first fluid from the first container, which is arranged on the first scale, into the supply path that extends to the outlet for the medical fluid,
operating the second pump to convey the second fluid from the second container, which is arranged on the second scale, into the supply path, to generate the mixture of the first and second fluids in the supply path,
measuring, by the sensor, the composition-related parameter of the mixture,
adjusting a pumping speed of at least one of the first and second pumps until the sensor measures a target value of the composition-related parameter,
determining, based on first and second output signals from the first and second scales, a relation between a first weight change of the first scale and a second weight change of the second scale while the sensor measures the target value, and
generating the medical fluid in the supply path, the generating comprising operating, based on the first and second output signals, the first and second pumps to achieve the relation between the first and second weight changes;
wherein the control device further comprises a signal interface, which is configured to output control signals for the first and second pumps and further configured to receive the first and second output signals from the first and second scales and a sensor signal representative of the composition-related parameter from said sensor.
